(19) United States
(12) Patent Application Publication
(10) Pub. No.: US 2010/0262245 A1
(43) Pub. Date: Oct. 14, 2010

(54) Intervertebral spacer

(76) Inventors: Arthur A. Alfaro, Colts Neck, NJ (US); Marc Burel, Perkasie, PA (US)

Correspondence Address: RALPH T. LILORE, 60 ROUTE 46 EAST, FAIRFIELD, NJ, 07004, US

(21) Appl. No.: 12/656,788

(22) Filed: Feb. 17, 2010

Related U.S. Application Data
(60) Provisional application No. 61/207,912, filed on Feb. 18, 2009.

(51) Int. Cl. A61F 2/44; A61B 17/58

(52) U.S. Cl. 623/17.16; 606/94

(57) ABSTRACT

Disclosed is a delivery system for an intervertebral spacer and a bone grafting material in the form of a unitary device which comprises a spacer disengagingly attached to a hollow handle. The handle comprises a chamber and bone grafting material-advancing means within the chamber for introducing the bone grafting material from the chamber into and around the spacer and the intervertebral spaces. The handle is disengageable from the spacer after serving its purpose of facilitating placement of the spacer and the discharge of the bone grafting material into the spacer and the intervertebral space.

The intervertebral spacer implant portion is attached to the handle portion at the exit port of the chamber, the point of attachment constituting the entry port of the intervertebral spacer implant portion.

The spacer has voids therein capable of receiving the bone grafting material from the chamber, and the chamber, the spacer implant portion voids, and the intervertebral spaces are in flow communication with each other whereby bone graft material expressed from the chamber is capable of entering the intervertebral spacer implant voids and the intervertebral spaces.

### CROSS REFERENCE TO RELATED APPLICATIONS

This application claims the benefit of U.S. Provisional Application No. 61/207,912 filed Feb. 18, 2009.

### STATEMENT REGARDING FEDERALLY SPONSORED RESEARCH OR DEVELOPMENT

(Not Applicable)

### REFERENCE TO A SEQUENCE LISTING A TABLE, OR A COMPUTER PROGRAM LISTING APPENDIX SUBMITTED ON A COMPACT DISC (SEE 37 CFR 1.52(e)(5)

(Not Applicable)

### FIELD OF THE INVENTION

This invention relates to the provision of surgical devices and more particularly to surgical devices for insertion of intervertebral spacer implants and delivery of bone grafting material into intervertebral spaces in surgical procedures.

### BACKGROUND OF THE INVENTION

It is often necessary in the correction of various spinal defects, to intervene and place exogenous devices between vertebrae in an effort to fuse adjacent vertebrae to each other. One particular modality is to introduce a solid material into the vertebral space following a surgical discectomy The solid material is pressure-fit into place between the opposing vertebral bodies so as to fix the device in place, and in essence, to encourage the two vertebrae to fuse. The intervertebral spacer usually contains voids that are packed with an osteoconductive and/or osteoinductive material (“biologic”, “biologic materials” or “bone grafting materials” herein) prior to insertion into the intervertebral space. The biologic material facilitates fusion of the two vertebrae to the spacer by the formation of bone to and through the intervertebral spacer from one vertebral body to the opposite vertebral body. It is important that the end plates of the superior and inferior vertebrae make good contact to the biologic material since bone does not span a gap or voids without the assistance of a conductive and inductive bridge.

The prior art is replete with spacers and inserters for inserting various spacers between vertebrae and for introducing various biologics thought to be useful in the fusion process into the spacer and the vertebral spaces.

The prior art spacers are of various sizes and shapes, but usually take the form of an anatomically suitable size and shape. They usually have voids or tunneling throughout to accommodate the biologic material needed to aid in the fusion of the spacer to the vertebrae and ultimately the fusion of adjacent vertebral bodies to each other.

Often, the spacer comes equipped with a detachable inserter which can be manipulated by the surgeon to facilitate the introduction of the spacer into the intervertebral space. Usually, the surgeon loads the intervertebral spacer with the biologic material with a syringe or manually. Once the spacer has been placed into position by the use of the inserter, the surgeon removes the detachable inserter leaving the spacer positioned in the patient. Often, the surgeon then introduces additional biologic material from a syringe or other loading device directly into and around the vertebral spacer so as to load-up the area with bone graft material. As far as we know, implant inserters have not heretofore been used to deliver the biologic material to the spacer nor have loading devices been used to insert implants.

A difficulty with the foregoing procedure when a spacer has been pre-loaded prior to insertion, is that as the surgeon is driving the intervertebral spacer into the intervertebral space, the biologic material that had been packed into the spacer will often fall out and/or settle. In addition, the irregularity of the surfaces of the vertebral end plates will cause cause gaps between the vertebral end plates, the biologic material and the intervertebral spacer. This prevents a complete and total fusion of the disparate materials thereby providing a potentially weakened fusion or non-fusion.

### BRIEF SUMMARY OF THE INVENTION

The present invention eliminates these and other disadvantages of the prior art devices. In the present invention, voids and gaps between the end plates of the vertebral body and the surfaces of the intervertebral spacer are filled by the virtually complete coverage at the surfaces thereof, with a suitable biologic product introduced via the unitary device of the invention. While we use the terms “biologic,” “biologic material” or “bone grafting material” herein to refer to the material used for osseointegration of the spacer with the vertebrae and for coverage of the surfaces and void spaces, we intend those terms to mean any materials which are or can be used in the spacer implant procedure. Such materials as cements and glues such as the cyanoacrylates may be employed even if such material is not normally considered a biological material. In addition, we use the term “spacer,” “implant” and “intervertebral spacer implant” interchangeably herein.

In general, the device of the present invention may be described as a delivery system in the form of a unitary device which comprises a spacer disengagingly attached to a hollow handle. The handle facilitates the introduction of the spacer by the surgeon into the intervertebral space. The handle comprises a chamber for delivery of appropriate biologic material, and material-advancing means within the chamber for introducing the bone grafting material from the chamber into and around the spacer and the intervertebral spaces. The handle is disengageable from the spacer after serving its purpose of facilitating placement of the spacer and the discharge of the bone grafting material into the spacer and the intervertebral space.

In a simple form, the intervertebral spacer of the device of the invention may be any spacer at all which satisfies the criteria of intervertebral spacers. It needs only to be attachable and detachable to a handle capable of containing a biologic material-advancing means such as an Archimedes screw, a plunger or syringe-type of system for moving the biologic material through the handle and into the spacer. The spacer itself will usually comprise voids and spaces which communicate with the chamber of the handle on the one hand and with the intervertebral spaces on the other and is part of the integrated unitary delivery system of the invention device. The terms “voids” and “spaces” are meant to be a generic description of empty spaces or compartments within the spacer to accommodate the bone grafting material, such as a hollowed out segment defining a chamber, tunnels, leaders and holes which lead into, and out of, the spacer itself and allow the flow of the bone grafting material into, out of, and around the spacer and the vertebral spaces. The handle not only provides the means for manipulating the spacer into position, but also acts as the means for introducing the biologic, into the spacer and the vertebral space. Thus, there is a direct line of flow through the handle into the voids of the spacer and out into the vertebral space.

In general, the system of the invention can be characterized as:

a surgical delivery device system for delivery of an intervertebral spacer implant and a bone graft material into an intervertebral space, comprising two delivery components, a first component in the form of a handle portion for manipulating the delivery device disengagingly attached to a second component constituting the intervertebral spacer implant portion,

the handle comprising a chamber capable of accommodating an effective amount of a bone graft material to be delivered, bone grafting material-advancing means in the chamber, and an exit port from the chamber for the delivery of the bone grafting material,

the intervertebral spacer implant portion having a point of attachment to the handle portion at the exit port of the chamber, the point of attachment constituting the entry port of the intervertebral spacer implant portion, whereby bone grafting material expressed from the exit port of the chamber enters said intervertebral spacer implant portion entry port,

the intervertebral spacer implant portion having voids therein capable of receiving the bone grafting material from the chamber, wherein

the chamber, the intervertebral spacer implant portion voids, and the intervertebral spaces being in flow communication with each other whereby bone graft material expressed from the chamber is capable of entering the intervertebral spacer implant voids and the intervertebral spaces.

In practice, the spacer is inserted surgically into the vertebral space and properly positioned therein using the handle as the inserter. The handle contains biologic material located in the chamber of the hollow handle. This material is then expressed via the material-advancing means, pushed through the chamber into the voids of the spacer and out into the intervertebral space. The excess material floods the space including the space between the surfaces of the spacer and the vertebrae giving a complete coverage or permeation of the interfaces. The handle is then disengaged from the spacer and the surgery appropriately terminated in the usual way.

As noted above, any spacer may be used provided it satisfies the requirements of the invention. The preferred spacer of the preferred device has the general structure shown in the Figures. A preferred structure includes, but is not limited to, a rectangular shape with rounded corners, or a kidney shape with varying degrees of curvature, oblong or round. The interior is hollow or open and most preferably divided along its long axis into two compartments. While two compartments are preferred, it is also contemplated to use only one compartment, and in some cases, though not preferred, no compartments at all. (Note: there are some spacers in the prior art which are solid but porous and can accommodate biologic material.) In three dimensions, the compartments accommodate the biologic material and are open to the top and bottom of the spacer thus providing access of the biologic to the intervertebral spaces. Most preferably, the spacer is shaped in a curvilinear fashion to approximate the shape of the vertebral body. Prior to use, the interior of the compartments is empty to allow for introduction of the biologic material. Associated with the individual compartments are a plurality of tunnels or leader lines which communicate with the intervertebral space and allow biologic material via pressure expression after the compartment space is full, to flow into the exterior space. A plurality of leader lines is provided, preferably spaced around the compartments, so as to promote even distribution of the biologic outflow within the space and around the top and bottom surface interfaces of the spacers and vertebrae and the sidewalls thereof.

The detachable or disengageable handle acts as an inserter of the spacer and comprises a hollow chamber to accommodate the biologic material to be added into the spacer. Any such handle may be used provided it can be engaged and disengaged with the spacer. Accordingly, a handle having a threaded end, generally into a reciprocally threaded spacer, is suitable. A pressure fit, a clip-on, a snap-on, or bayonet mount mechanisms are likewise suitable. Any disengageable means is suitable. The dimensions of the handle are such that sufficient biologic can be incorporated therein to fill the compartments and tunnels, and flow out into the interfaces between the compartments and the vertebrae to provide substantially complete coverage or coating of the interface surfaces. There are many handles or holders in the prior art which may be used in combination with the spacer to obtain the device of the invention. Therefore, those skilled in the art will at once be aware of many introducers and/or biologic delivery devices which can act as biologic material introducers. For example, the art has used syringes to introduce biologic materials into previously positioned spacers. Other devices such as those similar to caulking guns, ratcheting guns and the like may be employed. Any of the prior art forms which can be adapted to be disengagingly attachable to an appropriate spacer may be used to form the device of the invention provided the handle has a means of delivering biologic material and there is communication of spaces between the handle, the spacer and the intervertebral spaces.

The biologic material could include demineralized bone matrix (DBM) or some other osteogenic material like bone morphogenetic proteins (BMPs) or osteoconductive material which will become osseointegrated at the interface between the spacer and the vertebrae, thus forming a layer which will fuse the vertebral surface and the surface of the spacer. The spacer may be constructed of biologically acceptable material such as titanium, stainless steel, allograft bone, PEEK, or the like. Other cementitious materials may be employed in place of the DBM or along with the DBM such as the cyanoacrylates previously mentioned, hydroxyapatite, bone chips, various calcium phosphates, and the like. Biologically acceptable vehicles may also be employed to ensure the flowability of the material throughout the delivery system and anatomical spaces. In the preferred embodiment wherein DBM is used, the DBM is preferably supplied in a flowable composition comprising other vehicles or carriers well-known in the art for delivering DBM or other osteogenic or osteoconductive materials as aforementioned in the fusion of intervertebral bodies. Such materials as hyaluronic acid, collagen, glycerol, gelatin, poloxamer, calcium sulfate, lecithin, starch, any of the calcium phosphates, especially the triphosphate, Coralline-derived hydroxyapatite/calcium carbonate (HA/CC) composite are illustrative of the materials used in the art. These components and others maybe used with other cementitious materials as well. Those skilled in the art are fully aware of the compositions useful to achieve acceptable fusions when consideration is given to the functional cementitious materials used and the other components employed to provide suitable flowability. (See the compilation of commercially available materials hereinbelow).

### BRIEF DESCRIPTION OF THE DRAWINGS

FIG. 1 is a plan view of an embodiment of a simple form of the delivery device of the invention.

FIG. 2 is a plan view of the delivery device of FIG. 1 containing biologic material and in place in the anatomy of a patient.

FIGS. 3A, 3B, 3C, 3D, 3E, 4A, 4B, 4C, 4D, 5A, 5B, 5C, and 5D are various views of spacer handle embodiments of material-advancing means to be attachable to the spacer of the delivery device.

FIGS. 6 and 7 show in an elevation view, a prior art approach (FIG. 7) for delivering a biologic into the intervertebral spaces and the approach of the present invention (FIG. 6) of forcing DBM into the implant in situ as shown by the arrows.

FIGS. 8 and 9 shows a three dimensional perspective view of novel embodiments of the spacer of the invention.

### DETAILED DESCRIPTION OF THE INVENTION

The invention and various embodiments thereof will be better understood by reference to the drawings.

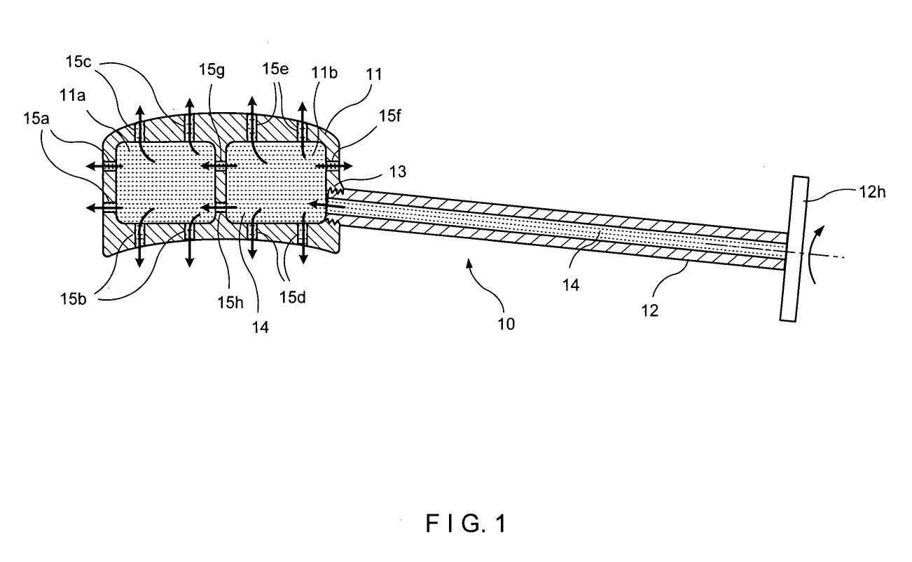

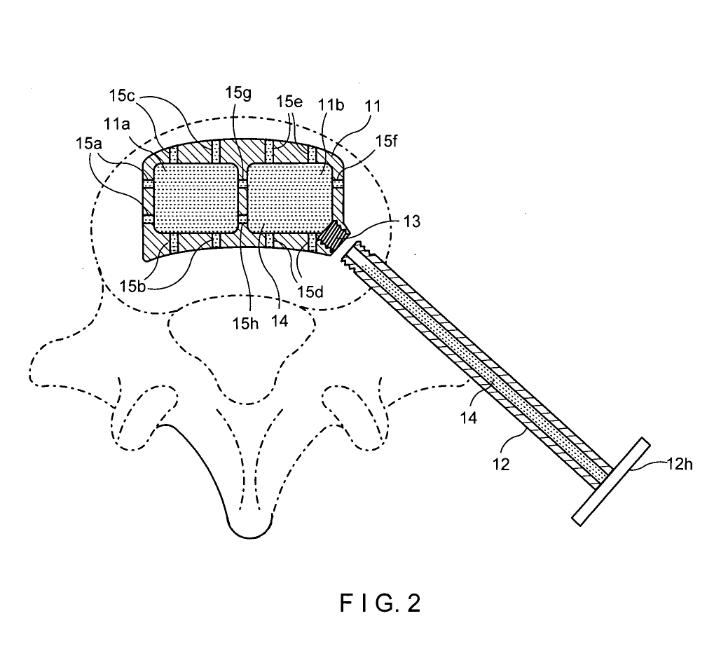

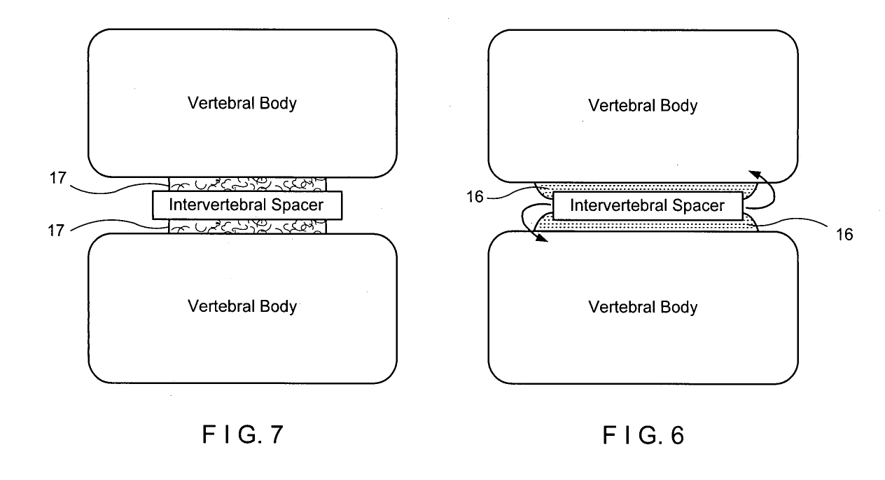

FIG. 1 shows the entire interbody grafting delivery system device 10 in a simple embodiment thereof wherein the spacer 11 is shown as part of the delivery system. FIG. 2 shows that device with the spacer 11 implanted in the anatomy of a patient. The device comprises spacer 11 which comprises open compartments 11(a) and 11(b), open at the top of the spacer and at the bottom at 15(i) and 15(j) (in FIG. 9) which are adapted to contain DBM or any other suitable biologic and communicate with the opposing vertebral surfaces to allow the biologic to flow into the space. DBM is preferred because it promotes bone growth and will fuse the surface of the spacer to the surface of the end plates more rapidly. Handle 12 is shown screwed into compartment 11(b) at 13 and is also shown to contain DBM 14 in the hollow portion of the handle and in compartments 11(a) and 11(b) and in tunnels 15(a),(b), (c), (d), (e) and (f), provided to allow for outflow of DBM or other biologic into the intervertebral space. The handle contains for example, an Archimedes screw to push and deliver demineralized bone matrix (DBM) to the implant site. See FIG. 6 at 16, and FIGS. 1 and 2. Compartments 11(a) and 11(b) are connected by tunneling 15(g) and (h) to allow biologic material to flow from the compartment of introduction to the other compartment and out into the intervertebral space. (See also FIG. 9, 15(g) and (h) combined). (See dotted line in FIG. 9). If desired, an additional screw hole can be placed at the side opposite 13 (see FIG. 1 and FIG. 9 at 15(k) and 15(l)) to provide flexibility for using the spacer and an additional tunnel outlet for biologic material.

For purposes of illustration, in use the surgeon implants the spacer into the correct location of the patient using the well-known techniques for intervertebral placements and observing all of the normal medical procedures attendant to this procedure. The surgeon then advances the DBM located in the handle 12(h) either by turning the handle or by using one of the embodiments described herein or any other present in the art. All of such would be suitable for supplying the material into the spacer.

Once the DBM is forced into the interior spacer compartment(s) and tunnels as shown in FIG. 2 at 11(a) and 11(b) and 15(a), (b), (c), (d), (e) and (f) respectively, with the DBM flowing through the compartments and into the vertebral spaces shown in FIG. 6 at 16, the handle is removed as by unscrewing it or pulling it away from its pressure fit or snap-on fit, and the procedure, for purposes of this invention, is terminated. The spacer of course, remains in place at the correct site between the vertebrae. It can be seen that by forcing the DBM into the implant in this manner, less gapping of DBM between the intervertebral spacer and the endplates of the vertebrae occurs leading to substantially increased fusion rates. This is to be compared to the situation existing using the current prior art approach as shown in FIG. 7 at 17 which may leave significant gaps between the spacer and the endplates of the inferior and superior vertebral bodies. Because of the nature of osteogenesis, bone will not grow across the gaps leaving a significantly weakened placement of the implant. The prior art approach involves placing the spacer and then attempting to introduce biologic material from a remote discharge device such as a syringe or delivery gun.

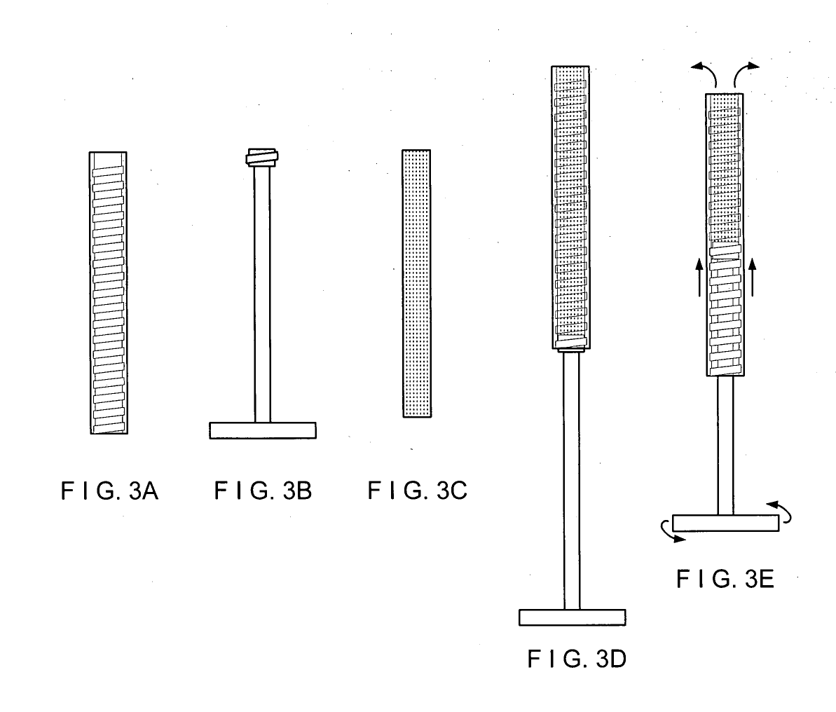

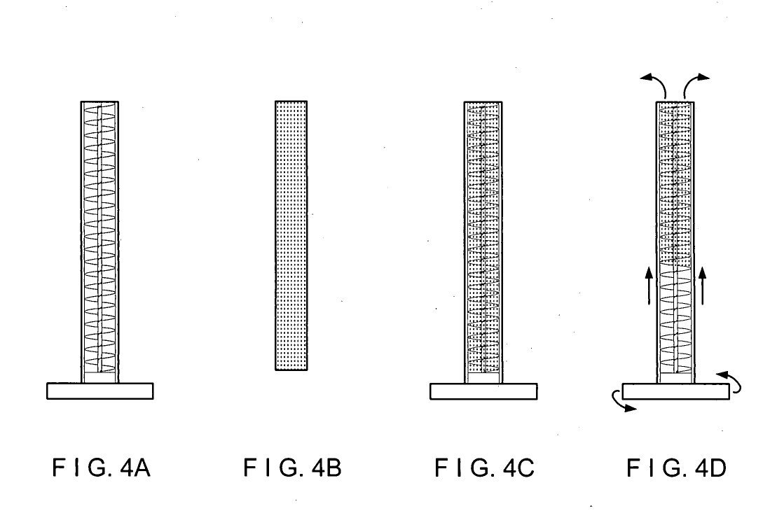

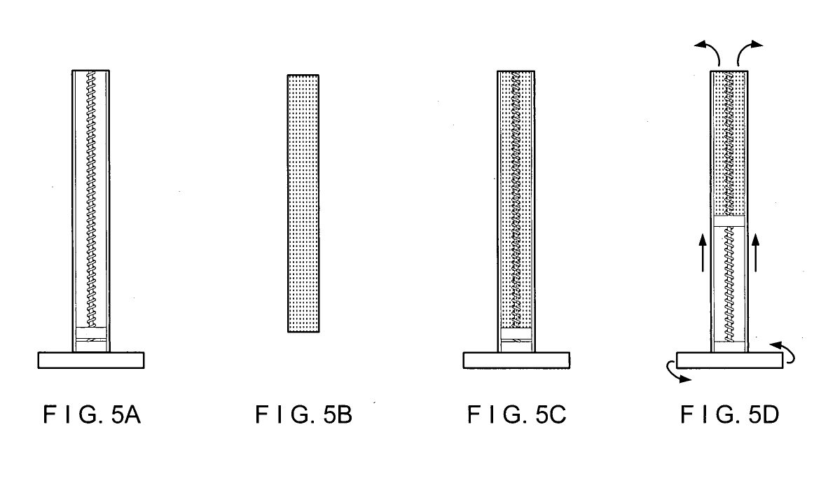

Various biologic delivery means in the form of handles, cannulae, syringes and the like, are shown in FIGS. 3, 4 and 5. The components of each Figure are designated respectively as FIGS. 3A, 3B, 3C, 3D and 3E; FIGS. 4A, 4B, 4C and 4D; and FIGS. 5A, 5B, 5C and 5D.

In FIGS. 3A-E,

Component 3A is a hollow cannula with internal grooves.
Component 3B is a driver/piston with tip that fits into the grooves of the cannula.
Component 3C is an optional bone biologic cartridge to load cannula.
Component 3D is the cannula assembled and loaded.

The device of FIG. 3E is fully assembled from the foregoing components, and shows at the arrows, that as the handle knob is turned, the piston is driven forward pushing out the biologic.

In FIGS. 4A-D,

Component 4A is a simple Archimedes screw.
Component 4B is a biologic loader.
Component 4C is the cannula assembled and loaded.

The device of FIG. 4D is fully assembled from the foregoing components, and shows at the arrows, that as the handle knob is turned, the screw is driven forward pushing out the biologic.

In FIGS. 5A-D,

Component 5A is a simple screw with a floating piston.
Component 5B is a biologic loader.
Component 5C is the cannula assembled and loaded.

The device of FIG. 5D is fully assembled from the foregoing components, and shows at the arrows, that as the handle knob is turned, the piston is driven forward pushing out the biologic.

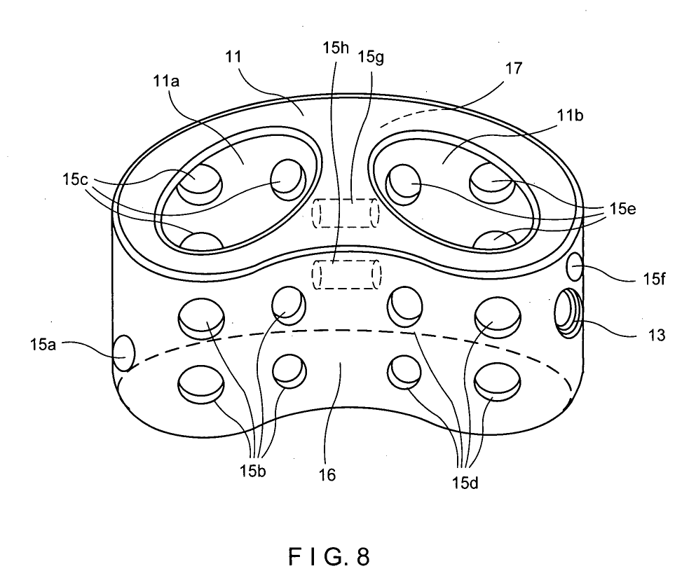

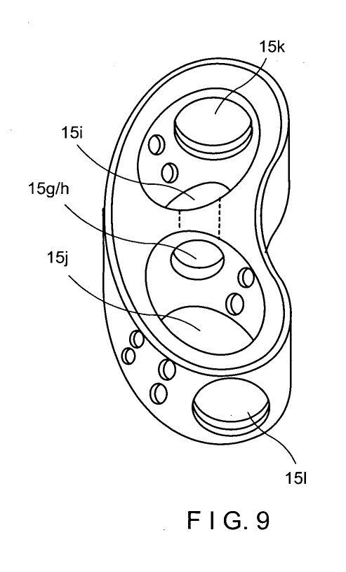

FIGS. 8 and 9 show the intervertebral (or interbody) spacer 11 in a preferred embodiment thereof. The spacer 11 comprises compartments 11(a) and 11(b) which descend as open spaces through the spacer. (See FIGS. 9, 15(i) and 15(j). The compartments are adapted to contain DBM or any other suitable biologic and comprise concave surface 16 and convex surface 17 (shown as an extended dotted line on the back of spacer 11 FIG. 8). The spacer in this embodiment is curvilinear in its bulk shape to approximate the anatomy. DMB is preferred because it promotes bone growth and will fuse the surface of the spacer to the surface of the endplates more rapidly. Handle 12 is not shown in these Figures but is intended to be screwed into compartment 11(b) at 13. Tunnels 15 (a), (b), (c), (d), (e) and (f) are provided to allow for outflow of DBM or other biologic into the intervertebral space (see FIG. 6 at 16), upon continued introduction of the biologic material from handle 12 in compartments 11(a) and 11(b). The lower tunnels 15(c) and 15(e), on the convex surface 17, leading from compartments 11(a) and 11(b) respectively, are not shown in this view. Tunnels 15(g) and (h) (shown separately in FIG. 8 and combined in FIG. 9) provide a biologic inter-connection between 11(b) and 11(a). Screw holes 15(k) and 15(l) show points of attachment for the holder, offering flexibility as to which side the surgeon prefers to use for delivery of the spacer.

Following is a listing of typical bone graft materials that are commercially available. AlloFuse™, a heat sensitive copolymer with DBM in the form of an injectable gel and putty available from AlloSource; ProOsteon®500R, a coralline-derived hydroxyapatite/calcium carbonate (HA/CC) composite in the form of a granules or block available from Biomet Osteobiologics.

InterGro®, a DBM in a lecithin carrier in the form of a paste, to be mixed with HA/CC composite granules; BonePlast®, a calcium sulfate with or without HA/CC in the form of a powder and setting solution both available from Biomet Osteobiologics.

HEALOS® Bone Graft Replacement, a mineralized collagen matrix in the form of a strip and, CONDUIT® TCP Granules, a 100% B-TCP in the form of Granules both available from DePuy Spine.

Opteform® a DBM and cortical cancellous chips in gelatin carrier in the form of a formable putty or dry powder, Optefil® a DBM suspended in gelatin carrier in the form of an injectable bone paste, dry powder; Optecure®, a DBM suspended in a hydrogel carrier in the form of a dry mix kit all available from Exactech, Optecure® + CCC a DBM and CCC suspended in a hydrogel carrier in the form of a dry mix kit available from Exactech; OpteMx™ a HA/TCP biphasic combination in the form of granules, sticks, rounded wedges, wedges and cylinders in several sizes available from Exactech.

Accell 100™, a DBM putty in the form of injectable putty; Accell Connexus® a DBM plus reverse phase medium in the form of a injectable putty; Accell TBM™, a total bone matrix, 100% preformed in the form of various sized strips; Integra Mozaik™, a 80% highly purified B-TCP, 20% highly purified type-1 collagen in the form of a strip and putty, all available from Integra Orthobiologics/(IsoTis Orthobiologics).

Optium DBM®, a DBM combined with glycerol carrier in the form of a formable putty (bone fibers) and injectable gel (bone particles); IC Graft Chamber® a DBM particles and cancellous chips in the form of a lyophilized, Cellect DBM® a DBM fibers and cancellous chips in the form of a specialized cartridge all available from LifeNet Health.

INFUSE® Bone Graft, a rhBMP-2 protein on an absorbable collagen sponge in the form of multiple kit sizes, MasterGraft® Granules a biphasic calcium phosphate in the form of Granules, MasterGraft® Matrix a calcium phosphate and collagen in the form of a compression resistant block available from Medtronic Spinal & Biologics, MasterGraft® Putty a calcium phosphate and collagen in the form of moldable putty, Progenix™ DBM Putty a DBM in Type-1 bovine collagen and sodium alginate in the form of an injectable putty, Osteofil® DBM a DBM in porcine gelatin in the form of injectable past and moldable strips all available from Medtronic Spinal & Biologics.

DBX®, a DBM in sodium hyaluronate carrier in the form of paste, putty mix and strip available from MTF/Synthes, NovaBone®, a Bioactive silicate in the form of a particulate and putty available from NovaBone/MTF, Vitoss® a 100% B-TCP and 80% B-TCP/20% collagen in the form of a putty, strip, flow, morsels and shapes available from Orthovita.

Grafton®, a DBM combined with Glycerol in the form of a formable putty, injectable gel, putty mixed with chips, flexible sheets and matrix available from Osteotech, Grafton Plus® a DBM combined with a starch carrier in the form of a paste available from Osteotech, BioSet™ a DBM combined with natural gelatin carrier in the form of an injectable paste, injectable putty, strips and blocks with cortical cancellous chips available from Regeneration Technologies.

VIAGRAF a DBM combined with glycerol in the form of a putty, paste, gel, crunch and flex available from Smith & Nephew, OP-1® Implant a rhBMP-7 with type 1 bone collagen in the form of a lyophilized powder reconstituted to form wet sand available from Stryker Biotech, OP-1® Putty a rhBMP-7 with type 1 bone collagen in the form of a lyophilized powder available from Stryker Biotech, Calstrux™, a tricalcium phosphate with carboxymethylcellulose in the form of a moldable putty available from Stryker Biotech, Norian® SRS® a calcium phosphate in the form of an injectable paste available from Synthes.

Norian® SRS® Fast Set Putty, a calcium phosphate in the form of a moldable putty, chronOS® a B-tricalcium phosphate in the form of granules, blocks and wedges available from Synthes.

Calceon®, 6 a calcium sulfate in the form of pellets available from Synthes, OSTEOSET® a surgical grade calcium sulfate in the form of pellets available from Wright Medical Technology, MIIG® X3 a high strength surgical grade calcium sulfate in the form of an injectable graft available from Wright Medical Technology, CELLPLEX® a tricalcium phosphate in the form of granules available from Wright Medical Technology, ALLOMATRIX® a DBM with/without CBM in surgical grade calcium sulfate powder in the form of an injectable/formable putty available from Wright Medical Technology, ALLOMATRIX® RCS a DBM with CACIPLEX™ Technology in surgical grade calcium sulfate powder in the form of a formable putty available from Wright Medical Technology, IGNITE® a DBM in surgical grade calcium sulfate powder to be mixed with bone marrow aspirate in the form of a percutaneous graft available from Wright Medical Technology.

CopiOs® Bone Void Filler a dibasic calcium phosphate and Type 1 collagen in the form of a sponge and paste available from Zimmer, CopiOs® Cancellous Bone Graft a bovine bone in the form of a cancellous chips, cancellous cubes and wedges available from Zimmer, Puros® Demineralized Bone Matrix a DBM putty in the form of a putty available from Zimmer.

What is claimed is:

1. A surgical delivery device system for delivery of an intervertebral spacer implant and a bone graft material into an intervertebral space comprising two delivery components, a first component in the form of a handle portion for manipulating said delivery device disengagingly attached to a second component constituting the intervertebral spacer implant portion which itself comprises a top surface and a bottom surface,
said handle comprising a chamber capable of accommodating an effective amount of said bone graft material to be delivered, bone grafting material advancing means in said chamber, and an exit port from said chamber for the delivery of said bone grafting material,
said intervertebral spacer implant portion having a point of attachment to said handle portion at the exit port of said chamber, said point of attachment constituting the entry port of said intervertebral spacer implant portion, whereby bone grafting material expressed from the exit port of said chamber enters said intervertebral spacer implant portion entry port,
said intervertebral spacer implant portion having voids therein capable of receiving said bone grafting material from said chamber,
said intervertebral spacer implant portion having holes therein designed to allow the complete filling of the spacer,
said intervertebral spacer implant portion having holes therein designed to allow the filling of the intervertebral space around and above the spacer
said chamber, said intervertebral spacer implant portion voids and said intervertebral spaces being in flow communication with each other whereby bone graft material expressed from said chamber is capable of entering said intervertebral spacer implant voids and said intervertebral spaces.

2. The device of claim 1 wherein the point of attachment of the handle and the spacer comprises a threaded attachment.

3. The device of claim 1 where in the point of attachment of the handle and the spacer comprises a pressure fit or a snap-on fit.

4. The device of claim 1 wherein the spacer comprises at least one interior compartment which opens to the outside at the top of the spacer and the bottom of the spacer.

5. The device of claim 4 wherein the interior compartment comprises a plurality of tunnels or leader lines leading to the outside of the spacer.

6. The device of claim 5 wherein the spacer is shaped to conform generally to the anatomy of the patient's intervertebral space.

7. The device of claim 6 wherein the spacer is generally kidney shaped.

8. The device of claim 5 wherein the handle is preloaded with a biologic material.

9. The device of claim 8 wherein the biologic material is a bone-grafting material.

10. The device of claim 9 wherein the bone-grafting material comprises one or more of DBM, bone chips, hydroxyapatite, a calcium phosphate, calcium sulfate, collagen, calcium carbonate, and a hyaluronic acid salt.

11. The device of claim 10 wherein the biological material is in the form of an injectable paste, gel, or putty and is flowable upon the application of pressure.

12. The device of claim 5 wherein the bone-grafting material-advancing means is a plunger.

13. The device of claim 5 wherein the bone-grafting material-advancing means is a syringe.

14. The device of claim 5 wherein the bone-grafting material-advancing means is an advancing screw.

15. The device of claim 10 wherein the bone-grafting material comprises DBM.

16. The device of claim 10 wherein the bone-grafting material comprises hydroxyapatite.

17. The device of claim 10 wherein the bone-grafting material comprises tricalcium phosphate.

18. The device of claim 10 wherein the bone-grafting material comprises bone morphogenic proteins.

19. The device of claim 10 wherein the bone-grafting material comprises bone chips.

20. The device of claim 10 wherein the bone-grafting material comprises collagen, hyaluronic acid or a salt thereof or carboxymethylcellulose.
